(19) United States
(12) Patent Application Publication
(10) Pub. No.: US 2003/0032011 A1
(43) Pub. Date: Feb. 13, 2003

(54) Method and apparatus for generating polycyclic fragments

(76) Inventor: Andrew Smellie, Gilroy, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 09/526,259

(22) Filed: Mar. 16, 2000

Related U.S. Application Data
(60) Provisional application No. 60/125,590, filed on Mar. 18, 1999.

(51) Int. Cl. C12Q 1/68; G06F 19/00; G01N 33/48; G01N 33/50; C12P 19/34; C12M 1/34

(52) U.S. Cl. 435/6; 435/91.2; 435/287.2; 702/20

(57) ABSTRACT

According to the present invention, automated techniques for determining molecular structures of polycyclic compounds are provided. More particularly, the present invention provides methods and apparatus for determining the structure of polycyclic chemical compounds using computer based methods to analyze subspecies of molecules, then combine the results from these analyses to determine the structure of the complete molecule.

### CROSS-REFERENCES TO RELATED APPLICATIONS

Further, this application makes reference to commonly owned, co-pending U.S. patent application Ser. No. 09/102,600, in the name of Andrew Smellie and Steven Teig, entitled, METHOD AND APPARATUS FOR CONFORMATIONALLY ANALYZING MOLECULAR FRAGMENTS, which is incorporated herein by reference in its entirety for all purposes.

### COPYRIGHT NOTICE

A portion of the disclosure of this patent document contains material which is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### BACKGROUND OF THE INVENTION

The present invention relates generally to the determination of molecular structure of polycyclic chemical compounds using computer based techniques to analyze subspecies of molecules, then combining the results from these analyses to determine the properties of the complete molecule.

Molecules have one or more three-dimensional structures. A molecule's structure determines its chemical, physical and bio-active properties. Scientists use a set of convenient parameters, such as bond length, bond angle and torsion angles, to describe the organization of atoms within a molecule that give rise to its molecular structure.

Researchers in the pharmaceutical field, for example, have sought for some time for a way to systematically analyze molecular structures of chemical compounds in order to determine their suitability as bioactive agents. A conformation is the spatial arrangement of the atoms in a molecule at any point in time that results from rotation of parts of the molecule about covalent bonds and the “bending” of bond angles, and “stretching” of bond lengths. Researchers in other fields also desire to search for chemical compounds having desirable attributes by analyzing molecules or fragments of molecules with a computer based method, rather than subjecting samples of the compounds to chemical analyses in a laboratory.

In a commonly owned, co-pending U.S. patent application Ser. No. 09/102,600, entitled, METHOD AND APPARATUS FOR CONFORMATIONALLY ANALYZING MOLECULAR FRAGMENTS, Smellie and Teig describe a method for determining conformational structures of molecules by searching conformer libraries for fragments that can be intersected to determine structures of entire molecules. While this is an important contribution to the field of drug research, there is no method described for automatically determining conformations of polycyclic ring structures that are part of the whole molecule.

What is needed is a method of determining conformations for a node, or polycyclic ring molecule, based upon information about the structure of its constituents.

### SUMMARY OF THE INVENTION

The present invention provides techniques for improved automated determination of molecular information. More particularly, the present invention provides methods and apparatus for determining the structure of polycyclic chemical compounds using computer based methods to analyze subspecies of molecules, then combine the results from these analyses to determine the structure of the complete molecule.

According to an embodiment of the present invention, a method for determining a conformation for a polycyclic molecular structure is provided. The method includes a variety of steps such as computationally decomposing the molecular structure into one or more constituent ring fragments. A step of determining, for each constituent ring, one or more ring conformers is also part of the method. In some embodiments, ring conformers can be stored in a stubbed form (i.e., retaining a subset of atoms around the ring to preserve context) in a database for retrieval by the method. The method also includes identifying atoms and bonds in common between the rings identified. These atoms and bonds in common can be determined based upon a context of the molecular structure. A step of identifying one or more torsions for each ring conformer is also included in the method. These torsions comprise the atoms and bonds in common between the rings identified in a previous method step. A step of determining an intersection of the ring conformers to form node conformers based upon the torsions identified in a previous method step is included in the method. The method can also include a step of determining from the node conformers, preferred node conformers, based upon a criterion. The combination of these steps can provide a method for determining a structure for polycyclic chemical compounds.

In another embodiment according to the present invention, a method can provide a minimal energy conformer as the preferred conformer for the node.

In a yet further embodiment of the present invention, a method for determining a conformation for a polycyclic molecular structure can provide an intersection of conformers based upon torsion of bonds in common between rings, as well as common stereochemistry based upon stereochemistry of atoms in common between rings. The method includes a variety of steps such as computationally decomposing the molecular structure into one or more substituent ring fragments. A step of determining for each substituent ring, one or more ring conformers is also part of the method. In some embodiments, conformers can be stored in a stubbed form in a database for retrieval by the method. The method also includes identifying atoms and bonds in common between the rings identified. These atoms and bonds in common can be determined based upon the context of the molecular structure. A step of identifying one or more torsions for each ring conformer is also included in the method. These torsions comprise the atoms and bonds in common between the rings identified in a previous method step. A step of identifying one or more stereochemical atoms in common for each ring is also included in the method. These atoms comprise the stereochemical atoms in common between the rings identified in a previous method step. A step of determining an intersection of the ring conformers to form node conformers based upon the torsions identified in a previous method step is included in the method.

A step of determining an intersection of the ring conformers to form node conformers based upon stereochemical atoms identified in a previous step can also be included in the method. The method can also include a step of determining from the node conformers, preferred node conformers, based upon a criterion. The combination of these steps can provide a method for determining a structure for polycyclic chemical compounds that can provide conformers based upon torsion of bonds in common between rings, as well as common stereochemistry based upon stereochemistry of atoms in common between rings. The combination of these steps can provide a method for determining a structure for polycyclic chemical compounds. In another embodiment according to the present invention, a method can provide a minimal energy conformer as the preferred conformer for the node.

In another aspect of the present invention, techniques for producing a library of conformers for substituent rings of a node are described. In a particular embodiment, a method for providing a conformer library of ring conformers includes steps of retrieving conformations of a simple ring and using them as starting conformations for the ring conformers. Atoms in the non-stub format ring that are not present in the simple ring are placed around the ring. Ring conformations can be refined using energy minimization, for example. The combination of these steps can provide a method for providing a conformer library of ring structures for analyzing polycyclic chemical compounds.

In another aspect of the present invention, techniques for producing a library of conformers of simple rings of a node are described. In a particular embodiment, a method for providing a conformer library of simple ring conformers includes steps of generating conformers of the node by an independent technique and extracting simple ring geometry from a context of the node. The extracting step can comprise steps of removing stubs, removing stereochemistry, removing cis and trans flags and replacing heavy atoms (e.g., atoms having molecular weights greater than a particular threshold value) with one or more equivalent atoms. Remaining simple ring atoms can be mapped to node atoms. A geometry of the node can be used to determine a geometry for the simple ring. The combination of these steps can provide a method for providing a conformer library of simple ring structures for analyzing polycyclic chemical compounds. Yet further, a combination of these steps can provide a method for maintaining a library of simple ring conformers which can be used to generate a library of ring conformers, from which node conformers can be determined.

Numerous benefits are achieved by way of the present invention over conventional techniques. In some embodiments, the present invention provides more automated methods of analyzing molecular structures using a computer than many of the manual techniques heretofore known. The present invention can also provide a library of torsions for compounds having polycyclic ring molecular structures. Some embodiments according to the invention can consider extended molecular contexts in determining a molecular structure based upon fragment conformers. Select embodiments according to the invention may be more robust than those using known techniques. Select embodiments according to the invention may be faster than those using known techniques. Select embodiments according to the invention may be more thorough than those using known techniques. These and other benefits are described throughout the present specification and more particularly below.

The invention will be better understood upon reference to the following detailed description and its accompanying drawings.

### BRIEF DESCRIPTION OF THE DRAWINGS

FIG. 1 is a block diagram of a system according to an embodiment according to the present invention;

FIGS. 2A-2D illustrate representative polycyclic ring conformations in embodiments according to the present invention;

FIG. 3 illustrates representative stereo-isomers;

FIGS. 4A-4C illustrate representative polycyclic ring structures and conformations during a plurality of processing steps in a representative embodiment according to the present invention;

FIGS. 5A-5B illustrate providing ring structures in libraries in a representative embodiment according to the present invention;

FIGS. 6A-6B illustrate simplified flow block diagrams of representative process steps for analyzing ring structures in a particular embodiment according to the present invention;

FIGS. 7A-7B illustrate simplified flow block diagrams of representative process steps for providing a conformer library of simple ring structures in a particular embodiment according to the present invention.

### DESCRIPTION OF THE SPECIFIC EMBODIMENTS

The present invention provides techniques for determining conformers of molecules based upon information about component molecular ring systems. Methods according to the present invention enable researchers and scientists to identify promising candidate compounds in the search for new and better substances.

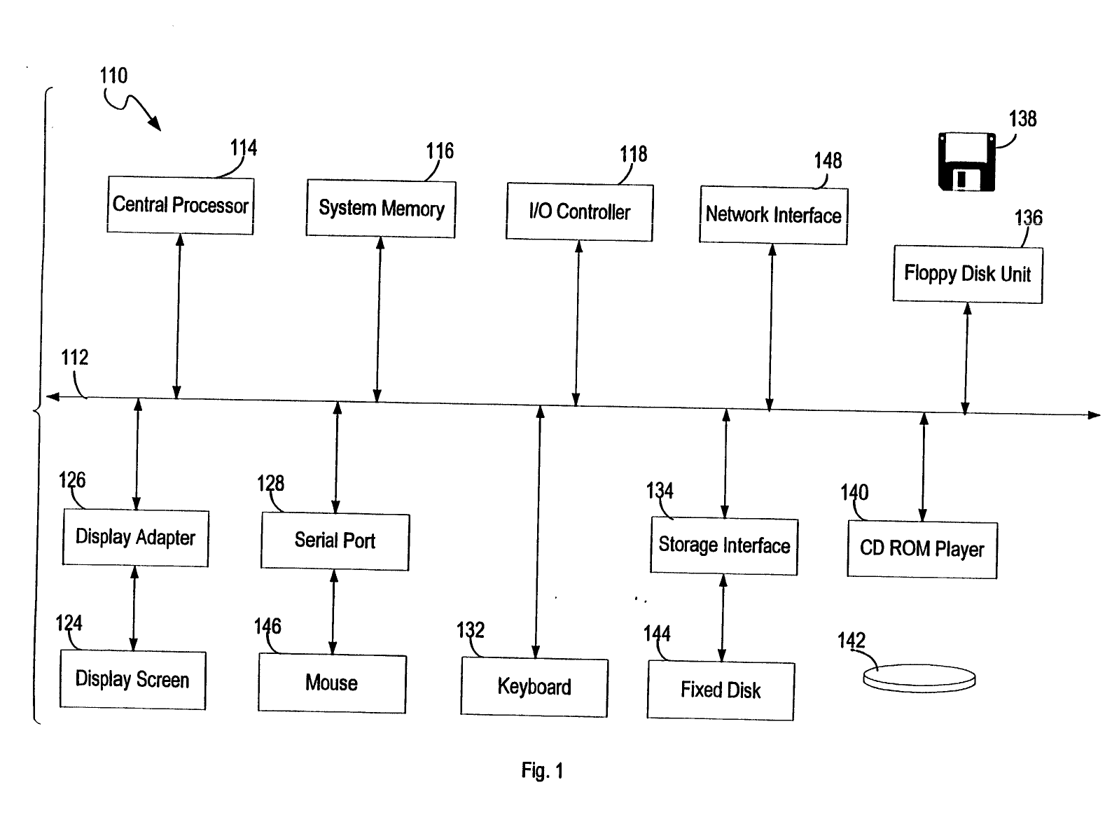

FIG. 1 depicts a block diagram of a host computer system 110 suitable for implementing the present invention. This diagram is merely an illustration and should not limit the scope of the claims herein. One of ordinary skill in the art would recognize other variations, modifications, and alternatives. Host computer system 110 includes a bus 112 which interconnects major subsystems such as a central processor 114, a system memory 116 (typically RAM), an input/output (I/O) controller 118, an external device such as a display screen 124 via a display adapter 126, a keyboard 132 and a mouse 146 via an I/O controller 118, a SCSI host adapter (not shown), and a floppy disk drive 136 operative to receive a floppy disk 138. Storage Interface 134 may act as a storage interface to a fixed disk drive 144 or a CD-ROM player 140 operative to receive a CD-ROM 142. Fixed disk 144 may be a part of host computer system 110 or may be separate and accessed through other interface systems. A network interface 148 may provide a direct connection to a remote server via a telephone link or to the Internet. Network interface 148 may also connect to a local area network (LAN) or other network interconnecting many computer systems. Many other devices or subsystems (not shown) may be connected in a similar manner. Also, it is not necessary for all of the devices shown in FIG. 1 to be present to practice the present invention, as discussed below. The devices and subsystems may be interconnected in different ways from that shown in FIG. 1. The operation of a computer system such as that shown in FIG. 1 is readily known in the art and is not discussed in detail in this application. Code to implement the present invention, may be operably disposed or stored in computer-readable storage media such as system memory 116, fixed disk 144, CD-ROM 140, or floppy disk 138.

System 110 is merely one example of a configuration that embodies the present invention. It will be readily apparent to one of ordinary skill in the art that many system types, configurations, and combinations of the above devices are suitable for use in light of the present disclosure. Of course, the types of system elements used depend highly upon the application.

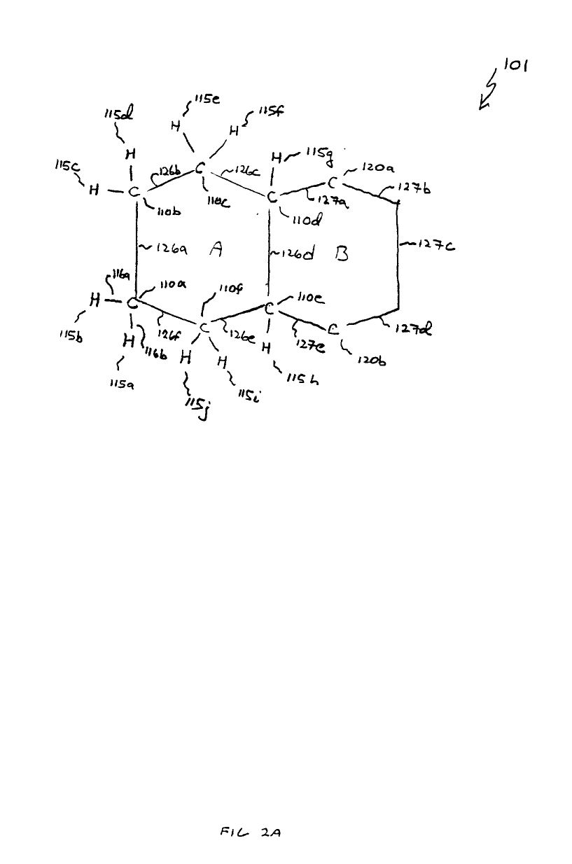

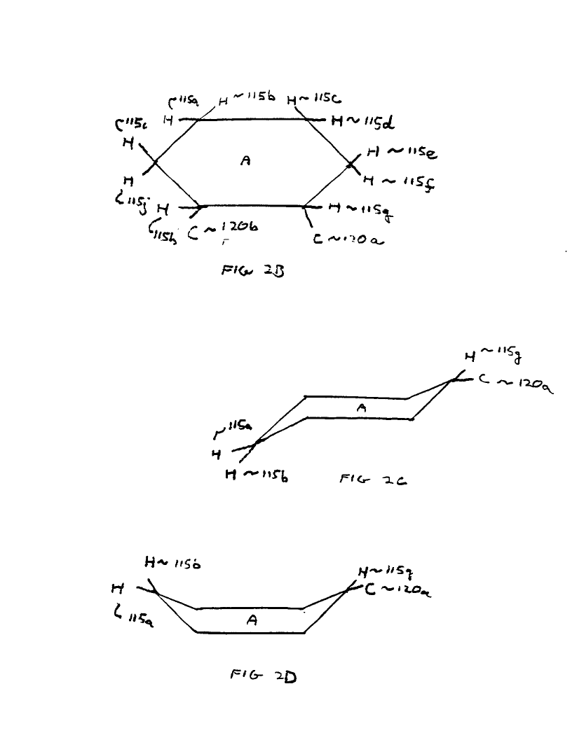

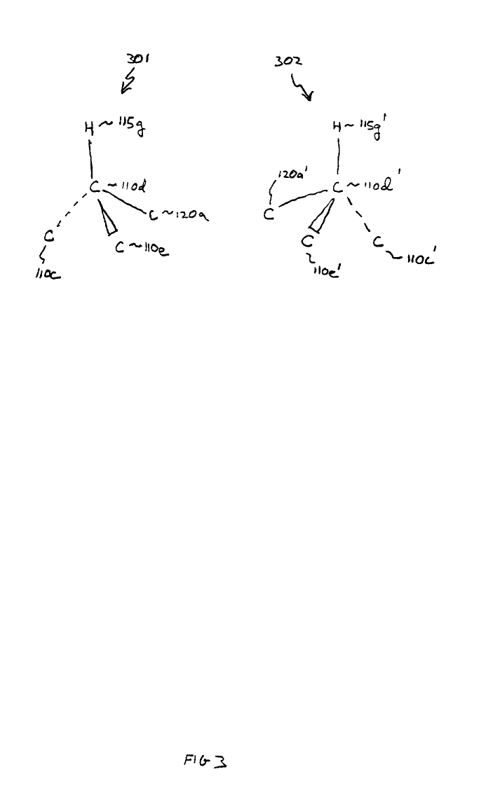

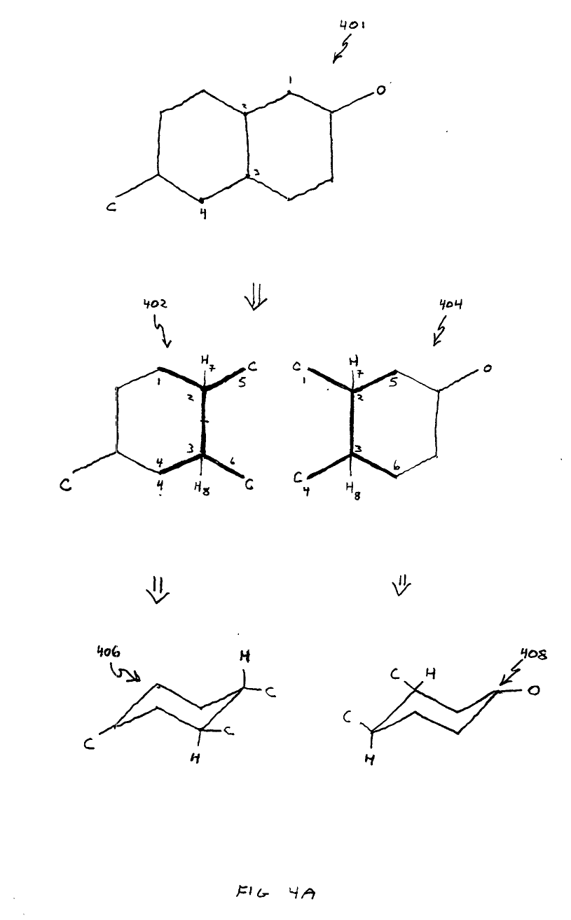

FIG. 2A depicts a molecular structure of a polycyclic molecule 101, in this example, polycyclic molecule 101 comprises a decalin molecule. This diagram is merely an illustration and should not limit the scope of the claims herein. One of ordinary skill in the art would recognize other variations, modifications, and alternatives. Molecule 101 comprises two ring structures, labeled A and B in FIG. 2A. For brevity, ring structure A is described in detail, as ring structure B is identical in structure to ring structure A. Ring structure A comprises carbon atoms 110a, 110b, 110c, 110d, 110e and 110f and hydrogen atoms 115a, 115b, 115c, 115d, 115e, 115f, 115g, 115h, 115i and 115j. Carbon atom l 10a is bound to hydrogen atoms 115a and 115b via chemical bonds 116a and 116b, respectively. Further, carbon atom 110a is bound to carbon atoms 110b and 110f in the ring via chemical bond 126a and chemical bond 126f, respectively, forming ring structure A with carbon atoms 110b, 110c, 110d, 110e and 110f. Similarly, carbon atoms 110b, 110c, 110d, 110e and 110f are also bound to hydrogen atoms, 115c, 115d, 115e, 115f, 115g, 115h, 115i and 115j respectively. Chemical bonds 126a through 126f are subject to torsion, whereby atoms in the ring can rotate relative to one another, yielding a plurality of conformers.

FIGS. 2B through 2D depict three potential conformations of ring A of molecule 101. Not all conformations will be possible for any given polycyclic molecular structure that contains ring A. FIG. 2B depicts a ring A of molecule 101 having a planar conformation. In the conformation depicted in FIG. 2B, bonds 126a, 126b, 126c, 126d, 126e and 126f are arranged such that carbon atoms 110a, 110b, 110c, 110d, 110e and 110f are positioned in the same plane, represented by the plane of the page. Hydrogen atoms 115a, 115b, 115c, 115d, 115e, 115f, 115g, 115h, 115i and 115j are positioned such that hydrogen atoms 115i 115b, 115d and 115f are above the plane of the page and hydrogen atoms 115j, 115a, 115c and 115e are below the plane of the page. Carbon atoms 120a and 120b are positioned such that carbon atom 120a is above the plane of the page and carbon atom 120b is below the plane of the page. This gives rise to the planar configuration of FIG. 2B.

FIG. 2C depicts ring A of molecule 101 in a “chair” conformation. In this conformation, hydrogen atoms 115a and 115b are positioned with the maximum distance from hydrogen atom 115g and carbon atom 120a.

FIG. 2D depicts ring A of molecule 101 in a “boat” conformation. In the conformation of FIG. 2D, hydrogen atoms 115a and 115b are positioned to be proximate to hydrogen atom 115g and carbon atom 120a. While FIGS. 2B-2D have illustrated potential confirmations for a six atom ring system, other conformers can be realized based upon the number and kinds of atoms joined in the ring. Additionally, not all conformations will be stable for a given ring in a polycyclic compound. Thus, the conformers depicted in FIGS. 2B-2D are intended to be illustrative and not restrictive of embodiments according to the present invention.

Torsions are an example of an internal coordinate representation of the molecule whereby with knowledge of the torsion angle about a bond coordinates of each atom rotated may be determined. In general, the representation of a conformation by its internal coordinates can be used to generate atomic coordinates for each atom in the molecule (modulo rotation and translation). There are many types of internal coordinates, well-known to those skilled in the art, that can be used in conjunction with the invention, but, in the preferred embodiment, torsion angles are the internal coordinate system used.

Experimental observations have shown that torsion angles are easier to change than bond angles, which in turn are easier to deform than bond lengths. This observation leads to the “fixed valence approximation” in which bond lengths and bond angles are assumed to be invariant, leaving only torsional angles as determinants of a molecule's structure. Even plastic molecular models hold bond angle and bond length fixed.

In many embodiments, the number of conformers in linear molecules, i.e., those with no rings, can be estimated for a particular molecule by calculating values for the expression N=s^{b}, where s is the number of samples of angular torsion to be examined for a bond, and b is the number of bonds whose torsion is to be sampled. For example, to calculate conformers for the decane molecule, which can be formed by breaking bonds 126d and 126e and adding hydrogens to carbon atoms 110d, 100e and 100f, in molecule 101 of FIG. 2A, there are 9 torsion angles representing rotation about bonds 126f, 126a, 126b, 126c, 127a, 127b, 127c, 127d and 127e. Given a sampling at 60, 180, and 300 degrees, 3 samples can be made per bond. Therefore, the number of conformers is 3⁹=19683. Calculating conformers for a simple molecule such as decane is computationally inexpensive. However, as the complexity of a molecule increases, the cost of calculating its conformers increases exponentially. For example, to calculate the conformers of a molecule having only 20 rotatable bonds and sampling each bond's torsion angle at 120 degree increments (i.e., 3 samples over 360 degrees of angle), N=s^{b}=3²⁰=3,486,784,401 conformers must be calculated. For rings systems, the situation is complicated by the ring closure constraint. In the case of decane, derived from decalin in FIG. 2A, carbon atoms 110d and 110e and carbon atoms 110f and 110e (i.e. at the ends of the chain), must be brought into proximity to close the ring. Thus, not every one of the conformers postulated above are feasible because most of them do not bring the ends of the chain into correct orientation to close the ring. Additional information regarding conformation and torsion may be found in Eliel, Stereochemistry of Carbon Compounds, Ch. 6, pp. 124-179 (1962), the entire contents of which are incorporated herein by reference for all purposes.

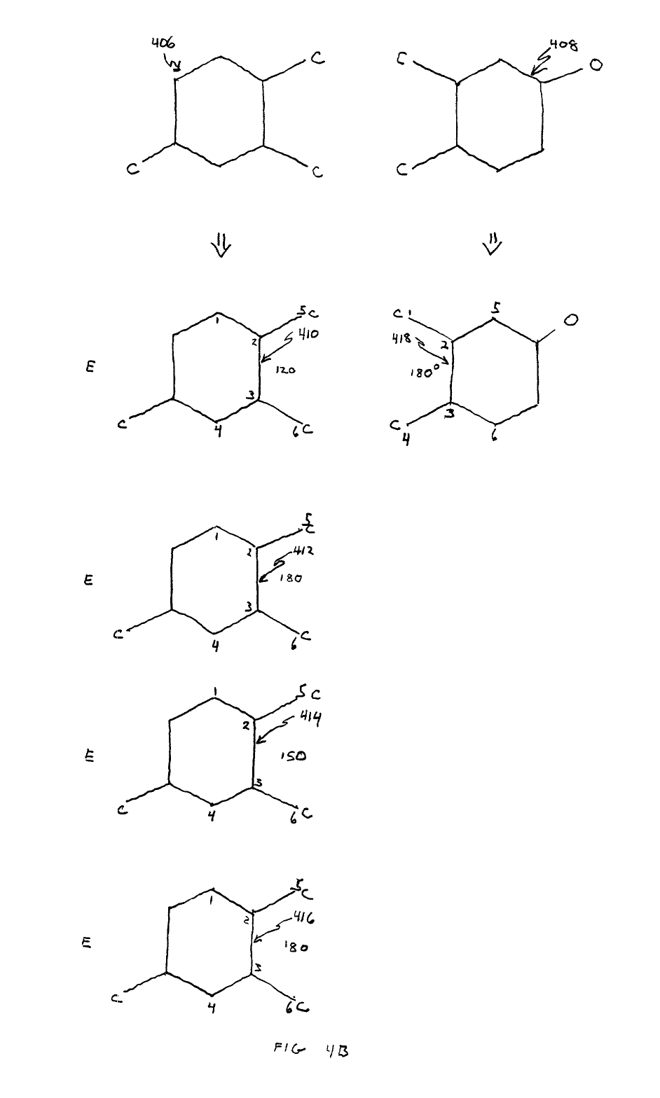

FIG. 3 illustrates stereoisomers 301 and 302, of a molecule, such as molecule 101 of FIG. 2A, in a particular embodiment according to the present invention. These diagrams are merely illustrations and should not limit the scope of the claims herein. One of ordinary skill in the art would recognize other variations, modifications, and alternatives.

Molecules are said to be stereoisomers if they possess identical structures, i.e., the same atoms bonded to one another in the same way, but differ in the manner that these atoms are arranged in space. These different structures are referred to as stereoisomers. Stereoisomers 301 and 302 are two molecules that are mirror images of one another. However, they are not superimposable (i.e. there is no rigid body translation and/or rotation that can superimpose atom pairs 115g-115g′, 120a-120a′, 110c-110c′, 110e-110e′ and 110d-110d′ simultaneously). Such molecules are said to possess opposite configurations or they are termed chiral molecules (chirality meaning “handedness” as the human hand is chiral) if the atoms 115g, 120a, 110e and 110c are distinguishable from each other. Physiological activity is often related to chiral configuration. For example, the left-rotating form of Adrenalin is over ten times more active in raising blood pressure than the right-rotating form of adrenalin.

FIG. 3 illustrates a representative stereoisomer 301 that comprises a carbon atom, such as carbon atom 110d, connected to a second carbon atom, such as carbon atom 110e. Carbon atom 110d is also connected to a third carbon atom 110c and a fourth carbon atom 120a. Additionally, carbon atom 110d is also connected to a hydrogen atom 115g. As illustrated, stereoisomer 301 is a three dimensional structure that has carbon atom 110e projecting out from the plane of the page, and carbon atom 110c projecting inward from the plane of the page. Stereoisomer 302 is a mirror image of stereoisomer 301. Stereoisomer 302 also comprises a central carbon atom 110d′ connected to a second carbon atom 110e′. Carbon atom 110d′ is also connected to carbon atom 110c′ and carbon atom 120a′. Finally, carbon atom 110d′ is also bonded to hydrogen atom 115g′. Note however, that although stereoisomer 302 is a mirror image of stereoisomer image 301, it is different in that carbon atom 110e′ projects out of the plane of the page and carbon atom 110c′ projects into the plane of the page, but nonetheless 301 and 302 are not superimposable

By convention, stereoisomer 302 is said to be counter clockwise, denoted by a commercial @ sign, while stereoisomer 301 is said to be clockwise, denoted by two commercial @ signs. This convention can be reversed in some embodiments. Other conventions can also be used without departing from the scope of the present invention. While the above has been described with reference to a particular molecular structure, that of molecule 101 of FIG. 2A, as illustrated by stereoisomers 301 and 302 of FIG. 3, many other compounds in nature exhibit stereo-isomeric properties. Thus, the above is intended to be representative and not restrictive of chemical compounds having this property.

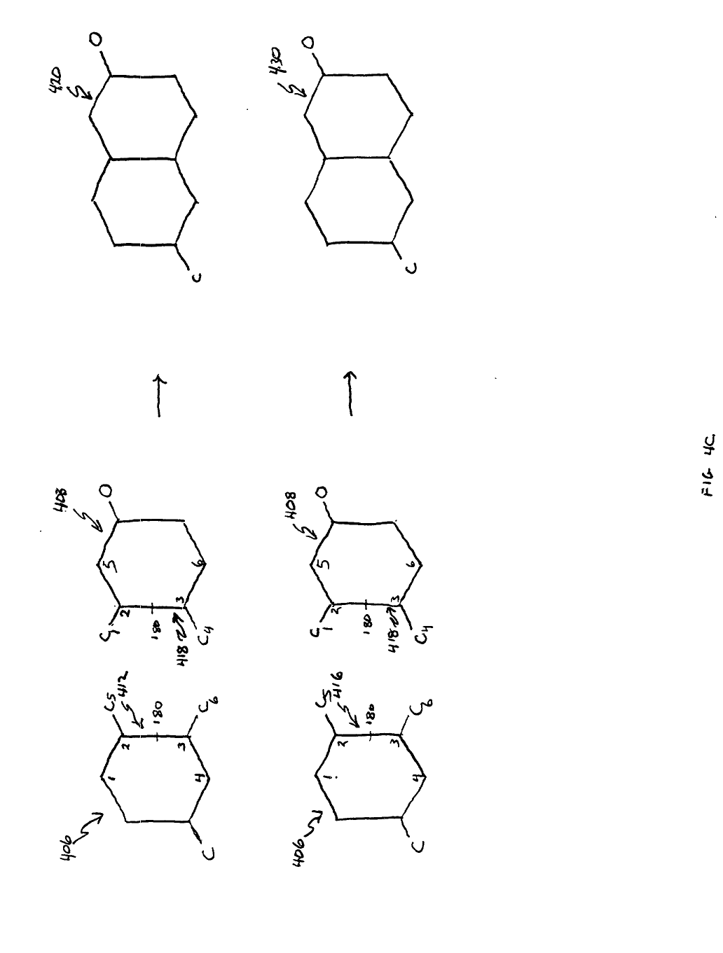

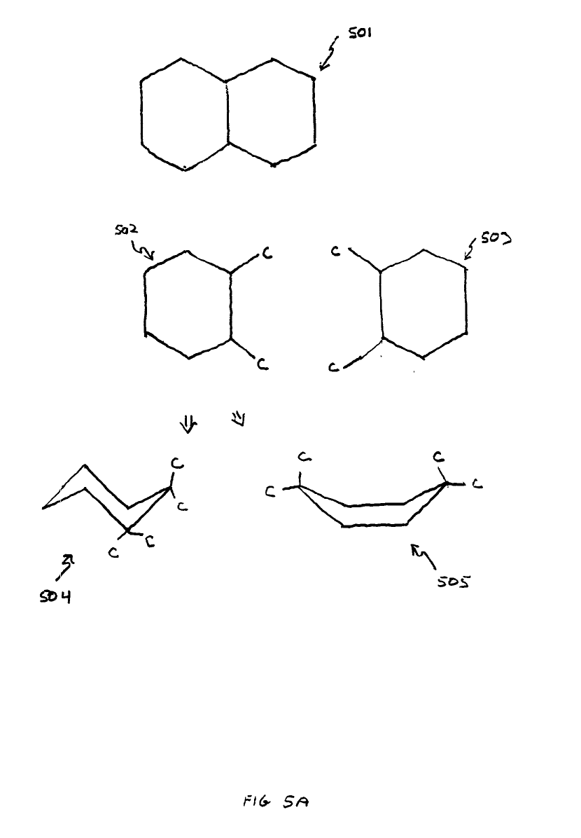

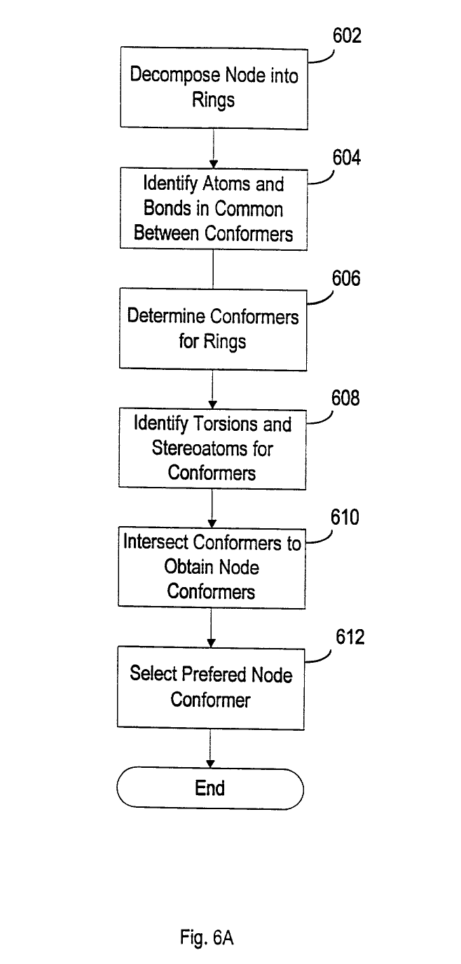

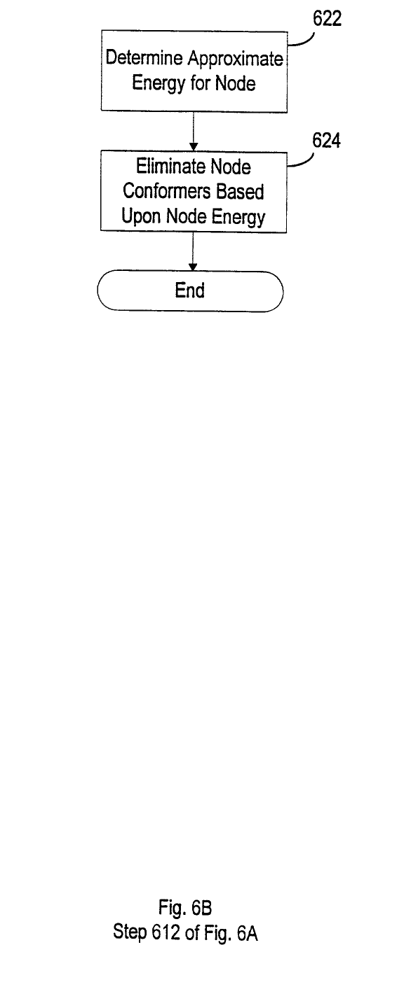

FIGS. 4A-4C illustrate representative polycyclic ring conformations and ring conformations from which they are constructed in various configurations while being processed by method steps of FIGS. 6A-6B in a particular embodiment according to the present invention. These diagrams are merely illustrations and should not limit the scope of the claims herein. One of ordinary skill in the art would recognize other variations, modifications, and alternatives. The conformations of FIGS. 4A-4C are discussed with reference to FIGS. 6A-6B below.

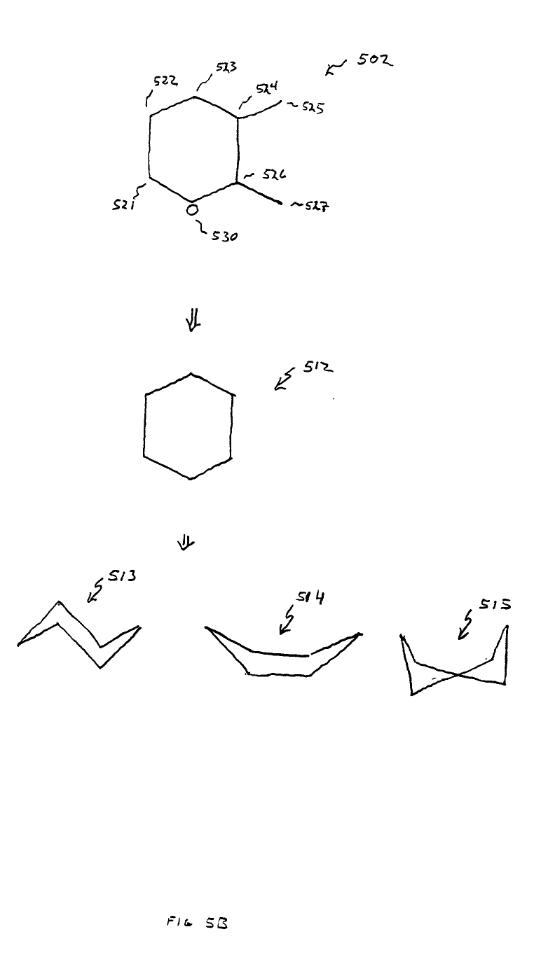

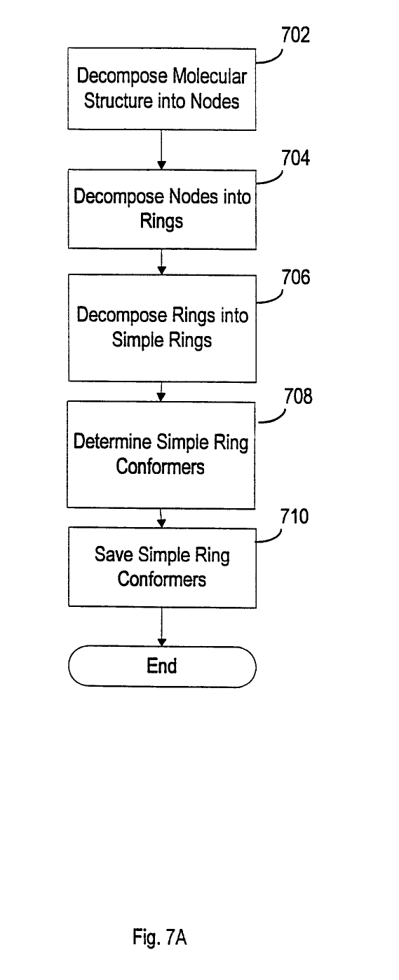

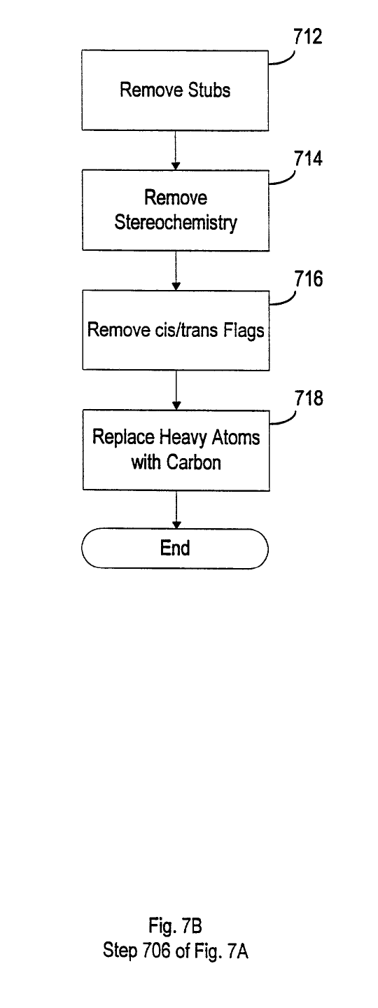

FIG. 5A illustrates providing ring conformations, such as the conformations, of FIGS. 4A-4C, suitable for storing in a ring library or the like in a representative embodiment according to the present invention. This diagram is merely an illustration and should not limit the scope of the claims herein. One of ordinary skill in the art would recognize other variations, modifications, and alternatives. FIG. 5A shows a node 501 being decomposed into substituent rings 502 and 503. Rings 502 and 503 include stubs and stereochemical information and cis/trans information. Conformations 504 and 505 are a representative set of a plurality of conformations for ring 502. Conformations 504 and 505 may be stored in a database for later retrieval. Process steps for providing ring conformations illustrated in FIG. 5A are described in FIG. 7A below.

FIG. 5B illustrates providing a plurality of simple ring conformers for determining ring conformations such as the conformations of FIGS. 4A-4C suitable for storing in a library or the like in a representative embodiment according to the present invention. This diagram is merely an illustration and should not limit the scope of the claims herein. One of ordinary skill in the art would recognize other variations, modifications, and alternatives. FIG. 5B depicts the determination of simple ring 512 from ring 502. Further, FIG. 5B depicts determining a plurality of generic simple ring conformers, such as simple ring conformers 513, 514 and 515, that can be used for constructing conformers for rings, such as conformers 406 and 408 of FIGS. 4A-4C. Process steps for providing simple ring conformations illustrated in FIG. 5B are described in FIGS. 7A and 7B below.

FIG. 6A illustrates a simplified flow block diagram of process steps in a method for determining a conformation for a polycyclic ring molecule substituent in a particular representative embodiment according to the present invention. This diagram is merely an illustration and should not limit the scope of the claims herein. One of ordinary skill in the art would recognize other variations, modifications, and alternatives. The molecule comprises a “node,” such as node 401 of FIG. 4A. Node 401 comprises a particular molecular structure, having one or more constituent ring structures, such as ring structures 402 and 404 of node 401. In a particular embodiment, this molecular structure is examined (“stubbed out”) one level out from the ring. In other words, effects arising from atoms bonded to atoms comprising the ring are considered, but the effects of atoms bonded to these atoms are not. However, other embodiments can be created that perform analysis two or more levels out from the ring without departing from the scope of the present invention. FIG. 6A illustrates a step 602 of decomposing the molecular structure of the node, such as node 401 of FIG. 4A, into a plurality of constituent rings, such as ring 402 and ring 404, of FIG. 4A.

Then, in a step 604, common atoms and bonds between the first ring 402 and the second ring 404 are identified. FIG. 4A illustrates atoms labeled 1-8 and their associated chemical bonds in common to ring 402 and ring 404.

Then, in step 606, for each substituent ring, one or more ring conformers having atoms and bonds in common to the rings identified in step 604 can be determined by searching in a library of ring conformers, previously constructed from a simple ring library, as illustrated by process steps of FIGS. 7A-7B with reference to the conformer libraries of FIGS. 5A-5B. FIG. 4A depicts a conformer 406 corresponding to ring 402 and conformer 408, corresponding to conformer 404. In many instances, more than one conformer can be found for a particular ring. In a present embodiment, conformers have the same atoms in the ring structure as the ring, but will have atoms and structures more than one level out eliminated. Other embodiments can use different “stubbing” atoms, or can be matched more than one level out from the ring without departing from the scope of the present invention.

In a step 608, for each ring conformer, one or more torsions can be identified. FIG. 4B illustrates conformers 406 and 408, derived in step 604, shown in a “top down” orientation and selected torsions determined for each of them in a particular representative embodiment according to the present invention. This diagram is merely an illustration and should not limit the scope of the claims herein. One of ordinary skill in the art would recognize other variations, modifications, and alternatives. FIG. 4B illustrates torsions 410, 412, 414 and 416 corresponding to conformer 406, and torsion 418 corresponding to conformer 408. Torsion 410 has an angle value of 120 degrees. Torsions 412 and 416 have angle values of 180 degrees (but the ring systems they are contained in differ in some other torsion in the ring). Torsion 414 has an angle value of 150 degrees. Torsion 418 has an angle value of 180 degrees.

Next, in a step 610, an intersection of ring conformers is determined from the torsions 410, 412, 414 and 416, corresponding to the first conformer 406, with the torsion 418, corresponding to the second conformer 408. The result of this intersection comprises node conformers. In this embodiment, torsions having a common angle value, survive the intersection. For example, FIG. 4B illustrates torsions 412, 416 having a 180 degree angle, as does torsion 418. Thus, in an embodiment using torsion angle as the intersection criterion, conformer pairs can be created having torsions 412-418 and torsions 416-418 by the intersection to form the node conformers. FIG. 4C illustrates an intersection operation for example conformers 406 and 408 of FIG. 4B. FIG. 4C illustrates a first combination of conformer 406, having a torsion value of 180 degrees, such as torsion 412 of FIG. 4B, and conformer 408, having a torsion value of 180 degrees, such as torsion 418 of FIG. 4B, to produce a node conformer, such as node conformer 420. Similarly, conformer 406, having a torsion value of 180 degrees, such as torsion 416 of FIG. 4B, and conformer 408, having a torsion value of 180 degrees, such as torsion 418 of FIG. 4B, can combine to produce node conformer 430 of FIG. 4C. In a presently preferable embodiment, a root means square (RMS) algorithm is used to superimpose the conformers according to a “best fit” of commonly shared atoms, numbered 1-6 in FIG. 4C, between the rings to determine the node conformers. RMS techniques are known to persons of ordinary skill in the art. For a detailed description of a particular example of an RMS technique, reference can be had to “S. Kearsley, “On the orthogonal transformation used for structural comparison”, Acta Cryst. 1989 A45 208,” the entire contents of which is incorporated herein by reference in its entirety for all purposes.

Then, in a step 612, a preferred node conformers are determined from the node conformers. The preferred node conformers are the conformation of the molecule within a user-defined energy threshold. In a presently preferable embodiment, determination of a preferred node conformer can be based on an energy minimization. However, other criteria can be used in various embodiments without departing from the scope of the present invention.

FIG. 6B illustrates simplified steps for selecting a preferred node conformer based on an energy criterion of step 612 of FIG. 6A in a presently preferable embodiment according to the present invention. FIG. 6B illustrates a step 622 of determining an approximate energy value for the node conformers, such as node conformer 420 and node conformer 430 produced in step 610, by summing a plurality of energy values stored for ring conformers in various particular torsions to arrive at an approximate energy for the node conformer. For example, an energy value 812 can correspond to conformer 406 having torsion 412. Similarly, an energy value 816 can correspond to conformer 406 having torsion 416, and an energy value 818 can correspond to conformer 408 having torsion 418, and so forth. Other methods can also be used to determine an approximate energy, such as look up tables, empirical methods and formulae and the like without departing from the scope of the present invention. Next, in a step 624, node conformers having energy values exceeding a selectable maximum value for the node are eliminated from the plurality of node conformers formed by the intersection of ring conformers in step 610 of FIG. 6A.

FIGS. 7A-7B illustrate simplified process steps for providing simple ring conformers for performing conformation analysis on polycyclic compounds, such as a representative compound 501 of FIGS. 5A-5B suitable for storing in libraries. These diagrams are merely an illustration and should not limit the scope of the claims herein. One of ordinary skill in the art would recognize other variations, modifications, and alternatives. FIG. 7A illustrates a step 702, wherein the molecular structure is decomposed into substituent nodes, and conformers are generated for these rings by use of a second conformer generation technique. In a presently preferable embodiment, an independent conformer generation technique can be used so as to prevent circularity in the method. we are using node conformers to build simple ring conformers, which in turn are being used to build ring conformers, which in turn are being used to build node conformers. In many embodiments, an expected number of unique simple rings can be comparatively small, a simple ring library can be built for a relatively small number of nodes until conformations of simple rings in most likely shapes have been determined. Such libraries can then be used to build node conformers for many different nodes (not merely the ones used to train the simple ring library). In a particular embodiment, distance geometry and energy minimization techniques can be used to limit the number of conformers stored in the library. Distance geometry algorithms are known to persons of ordinary skill in the art. For a detailed description of a representative example of a distance geometry technique, reference can be had to “Crippen and Havel, Distance Geometry and Conformational Calculations, Research Studies Press, Wiley Press (1981),” the entire contents of which is incorporated herein by reference in its entirety for all purposes. Then, in a step 704, the node is decomposed into one or more substituent rings. For example, node 501 of FIG. 5A is decomposed into substituent rings 502 and 503. In a presently preferable embodiment, such rings can comprise stubs, stereochemical information and cis/trans information. Then, in a step 706, rings, such as rings 502 and 503, are decomposed into simple rings, such as simple ring 512 of FIG. 5B. Such simple rings are produced by the process illustrated in FIG. 7B in which stubs, stereochemisty and cis/trans flags of rings are removed. In a step 708, simple ring conformers are obtained by extracting the geometry of the corresponding atoms of the original node of step 702. Finally, in step 710, the simple ring conformers can be stored in a database for later retrieval

FIG. 7B illustrates steps for extracting simple rings from a context of a ring of step 706 of FIG. 7A in a particular embodiment according to the present invention. Simple rings, such as ring 512 of FIG. 5B, can be derived from rings, such as ring 502 of FIG. 5A. Ring 502, as illustrated in FIG. 5B, is comprised of carbon atoms 521, 522, 523, 524, 526 and an oxygen atom 530. FIG. 7B illustrates a step 712 of removing stubs from the ring 502, by deleting the atoms and bonds of the stub. Then, in a step 714, stereochemistry is removed from the ring, by deleting the stereochemical data for simple ring atoms. Next, in a step 716, cis and trans flags are removed from the rings by deleting cis/trans information for simple ring bonds. Then, in a step 718, heavy atoms, i.e., atoms of elements having molecular weights greater than that of hydrogen, are replaced by carbon atoms. Thus, in the example of FIG. 5B, oxygen atom 530 of ring 502 is replaced by a carbon atom in simple ring 512.

### CONCLUSION

Although the above has generally described the present invention according to specific embodiments, the present invention has a much broader range of applicability. In particular, the present invention is not limited to a particular kinds of compounds, but can be applied to any polycyclic ring structure where an improved or optimized analysis is desired. Thus, in some embodiments, the techniques of the present invention could provide conformations for many different kinds of substances, having any number and types of ring structured substituents. Of course, one of ordinary skill in the art would recognize other variations, modifications, and alternatives.

What is claimed is:

1. A method for determining a conformation for a polycyclic ring molecule, said molecule comprising a node, said node having a molecular structure comprising at least one of a plurality of ring structures, said method comprising:
decomposing said molecular structure into a plurality of substituent rings, including a first ring and a second ring;
for each substituent ring, determining at least one of a plurality of ring conformers, including, a first ring conformer and a second ring conformer;
identifying atoms and bonds in common between said first ring conformer and said second ring conformer, said atoms and bonds in common determined based upon a context of said molecular structure;
for each ring conformer, identifying at least one of a plurality of torsions, including a first plurality of torsions, corresponding to said first ring conformer, and a second plurality of torsions, corresponding to said second ring conformer, said torsions comprising said atoms and bonds in common between said first ring conformer and said second ring conformer;
determining an intersection of said ring conformers, to form node conformers, said intersection comprising conformers having a value of torsion in common;
determining from said plurality of node conformers, preferred node conformers, said preferred node conformers comprising said conformation of said molecule.

2. The method of claim 1 wherein said torsion comprises a quartet of atoms.

3. The method of claim 1 wherein said determining at least one of a plurality of ring conformers further comprises:
identifying in a library at least one conformer, said conformer having the same molecular structure as the corresponding substituent ring;
retrieving said conformer from said library.

4. The method of claim 3 wherein said library further contains an energy level of said ring conformer, and wherein said retrieving said conformer from said library further comprises retrieving said energy level along with said conformer, said determining from said plurality of node conformers, a preferred node conformer further comprising:
determining a sum of said stored energy levels for each node conformer, said sum comprising an approximate energy level;
eliminating from said plurality of node conformers, ones having said approximate energy level exceeding a user-specified energy level.

5. The method of claim 1 wherein said intersection further comprises determining a root mean square (RMS) of fit between ring conformers for said atoms in common.

6. The method of claim 3 wherein said library further contains an indicator of stereo-isomerism of said ring conformer, and wherein said retrieving said conformer from said library further comprises retrieving said indicator of stereo-isomerism along with said conformer, said determining an intersection of said ring conformers, to form node conformers further comprising:
determining conformers having a value of said indicator of stereo-isomerism in common.

7. A method for determining a conformation for a polycyclic ring molecule, said molecule comprising a node, said node having a molecular structure comprising at least one of a plurality of ring structures, said method comprising:
decomposing said molecular structure into a plurality of substituent rings, including a first ring and a second ring;
for each substituent ring, identifying in a library at least one conformer, said conformer having the same molecular structure as the corresponding substituent ring, including a first ring conformer and a second ring conformer;
retrieving from said library at least one of a plurality of ring conformers, including said first ring conformer and said second ring conformer;
for each ring conformer, retrieving from said library an energy level associated with said ring conformer, including a first energy level and a second energy level;
for each ring conformer, retrieving from said library an indicator of stereo-isomerism of said ring conformer, including a first indicator of stereo-isomerism and a second indicator of stereo-isomerism;
identifying atoms and bonds in common between said first ring conformer and said second ring conformer, said atoms and bonds in common determined based upon a context of said molecular structure;
for each ring conformer, identifying at least one of a plurality of torsions, including a first plurality of torsions, corresponding to said first ring conformer, and a second plurality of torsions, corresponding to said second ring conformer, said torsions comprising said atoms and bonds in common between said first ring conformer and said second ring conformer;
determining an intersection of said ring conformers, to form node conformers, said intersection comprising conformers having a value of torsion and a value of said indicator of stereo-isomerism in common;
determining an approximate energy level for each node conformer, said energy level comprising a sum of energy levels for individual conformers retrieved from said library;
eliminating from said plurality of node conformers, ones having said approximate energy level exceeding a user-specified energy level to arrive at preferred node conformers, said preferred node conformers comprising said conformation of said molecule.

8. The method of claim 7 wherein said torsion comprises a quartet of atoms.

9. The method of claim 7 wherein said intersection further comprises determining a root mean square (RMS) of fit between ring conformers for said atoms and bonds in common.

10. The method of claim 7 wherein said indicator of stereo-isomerism comprises a −1 for counterclockwise and a −2 for clockwise.

11. A computer program product for determining a conformation for a polycyclic ring molecule, said molecule comprising a node, said node having a molecular structure comprising at least one of a plurality of ring structures, said product comprising:
code for decomposing said molecular structure into a plurality of substituent rings, including a first ring and a second ring;
code for for each substituent ring, determining at least one of a plurality of ring conformers, including a first ring conformer and a second ring conformer;
code for identifying atoms and bonds in common between said first ring conformer and said second ring conformer, said atoms and bonds in common determined based upon a context of said molecular structure;
code for for each ring conformer, identifying at least one of a plurality of torsions, including a first plurality of torsions, corresponding to said first ring conformer, and a second plurality of torsions, corresponding to said second ring conformer, said torsions comprising said atoms and bonds in common between said first ring conformer and said second ring conformer;
code for determining an intersection of said ring conformers, to form node conformers, said intersection comprising conformers having a value of torsion in common;
code for determining from said plurality of node conformers, preferred node conformers, said preferred node conformers comprising said conformation of said molecule; and
a computer memory for containing said codes.

12. The computer program product of claim 11 wherein said torsion comprises a quartet of atoms.

13. The computer program product of claim 11 wherein said code for determining at least one of a plurality of ring conformers further comprises:
code for identifying in a library at least one conformer, said conformer having the same molecular structure as the corresponding substituent ring;
code for retrieving said conformer from said library.

14. The computer program product of claim 13 wherein said library further contains an energy level of a torsion of said ring conformer, and wherein said retrieving said conformer from said library further comprises retrieving said energy level along with said conformer, said determining from said plurality of node conformers, a preferred node conformer further comprising:
code for determining a sum of said stored energy levels for each node conformer, said sum comprising an approximate energy level;
code for eliminating from said plurality of node conformers, ones having said approximate energy level exceeding a user-specified energy level.

15. The computer program product of claim 11 wherein said code for determining an intersection further comprises code for determining a root mean square (RMS) of fit between ring conformers for said atoms and bonds in common.

16. The computer program product of claim 13 wherein said library further contains an indicator of stereo-isomerism of said ring conformer, and wherein said code for retrieving said conformer from said library further comprises code for retrieving said indicator of stereo-isomerism along with said conformer, said code for determining an intersection of said ring conformers, to form node conformers further comprising:
code for determining conformers having a value of said indicator of stereo-isomerism in common.

17. A computer program product for determining a conformation for a polycyclic ring molecule, said molecule comprising a node, said node having a molecular structure comprising at least one of a plurality of ring structures, said computer program product comprising:
code for decomposing said molecular structure into a plurality of substituent rings, including a first ring and a second ring;
code for for each substituent ring, identifying in a library at least one conformer, said conformer having the same molecular structure as the corresponding substituent ring, including a first ring conformer and a second ring conformer;
code for retrieving from said library at least one of a plurality of ring conformers, including said first ring conformer and said second ring conformer;
code for for each ring conformer, retrieving from said library an energy level associated with said ring conformer, including a first energy level and a second energy level;
code for for each ring conformer, retrieving from said library an indicator of stereo-isomerism of said ring conformer, including a first indicator of stereo-isomerism and a second indicator of stereo-isomerism;
code for identifying atoms and bonds in common between said first ring conformer and said second ring conformer, said atoms and bonds in common determined based upon a context of said molecular structure;
code for for each ring conformer, identifying at least one of a plurality of torsions, including a first plurality of torsions, corresponding to said first ring conformer, and a second plurality of torsions, corresponding to said second ring conformer, said torsions comprising said atoms and bonds in common between said first ring conformer and said second ring conformer;
code for determining an intersection of said ring conformers, to form node conformers, said intersection comprising conformers having a value of torsion and a value of said indicator of stereo-isomerism in common;
code for determining an approximate energy level for each node conformer, said energy level comprising a sum of energy levels for individual conformers retrieved from said library;
code for eliminating from said plurality of node conformers, ones having said approximate energy level exceeding a user-specified energy level to arrive at preferred node conformers, said preferred node conformers comprising said conformation of said molecule; and
a computer readable storage medium for containing said codes.

18. The computer program product of claim 17 wherein said torsion comprises a quartet of atoms.

19. The computer program product of claim 17 wherein said code for determining an intersection further comprises code for determining a root mean square (RMS) of fit between ring conformers for said atoms and bonds in common.

20. The computer program product of claim 17 wherein said indicator of stereo-isomerism comprises a −1 for counterclockwise and a −2 for clockwise.

21. An apparatus for determining a conformation for a polycyclic ring molecule, said molecule comprising a node, said node having a molecular structure comprising at least one of a plurality of ring structures, said apparatus comprising:
a memory;
a display;
a bus, said bus connecting said memory and said display to a processor, said processor operatively disposed to perform the following:
decomposing said molecular structure into a plurality of substituent rings, including a first ring and a second ring;
for each substituent ring, determining at least one of a plurality of ring conformers, including a first ring conformer and a second ring conformer;
identifying atoms and bonds in common between said first ring conformer and said second ring conformer, said atoms and bonds in common determined based upon a context of said molecular structure;
for each ring conformer, identifying at least one of a plurality of torsions, including a first plurality of torsions, corresponding to said first ring conformer, and a second plurality of torsions, corresponding to said second ring conformer, said torsions comprising said atoms and bonds in common between said first ring conformer and said second ring conformer;
determining an intersection of said ring conformers, to form node conformers, said intersection comprising conformers having a value of torsion in common;
determining from said plurality of node conformers, preferred node conformers, said preferred node conformers comprising said conformation of said molecule.

22. The apparatus of claim 21 wherein said torsion comprises a quartet of atoms.

23. The apparatus of claim 21 wherein said determining at least one of a plurality of ring conformers further comprises:
identifying in a library at least one conformer, said conformer having the same molecular structure as the corresponding substituent ring;
retrieving said conformer from said library.

24. The apparatus of claim 23 wherein said library further contains an energy level of a torsion of said ring conformer, and wherein said retrieving said conformer from said library further comprises retrieving said energy level along with said conformer, said determining from said plurality of node conformers, a preferred node conformer further comprising:
determining a sum of said stored energy levels for each node conformer, said sum comprising an approximate energy level;
eliminating from said plurality of node conformers, ones having said approximate energy level exceeding a user-specified energy level.

25. The apparatus of claim 21 wherein said intersection further comprises determining a root mean square (RMS) of fit between ring conformers for said atoms and bonds in common.

26. The apparatus of claim 23 wherein said library further contains an indicator of stereo-isomerism of said ring conformer, and wherein said retrieving said conformer from said library further comprises retrieving said indicator of stereo-isomerism along with said conformer, said determining an intersection of said ring conformers, to form node conformers further comprising:
determining conformers having a value of said indicator of stereo-isomerism in common.

27. An apparatus for determining a conformation for a polycyclic ring molecule, said molecule comprising a node, said node having a molecular structure comprising at least one of a plurality of ring structures, said apparatus comprising:
a memory;
a display;
a bus, said bus connecting said memory and said display to a processor, said processor operatively disposed to perform the following:
decomposing said molecular structure into a plurality of substituent rings, including a first ring and a second ring;
for each substituent ring, identifying in a library at least one conformer, said conformer having the same molecular structure as the corresponding substituent ring, including a first ring conformer and a second ring conformer;
retrieving from said library at least one of a plurality of ring conformers, including said first ring conformer and said second ring conformer;
for each ring conformer, retrieving from said library an energy level associated with said ring conformer, including a first energy level and a second energy level;
for each ring conformer, retrieving from said library an indicator of stereo-isomerism of said ring conformer, including a first indicator of stereo-isomerism and a second indicator of stereo-isomerism;
identifying atoms and bonds in common between said first ring conformer and said second ring conformer, said atoms and bonds in common determined based upon a context of said molecular structure;
for each ring conformer, identifying at least one of a plurality of torsions, including a first plurality of torsions, corresponding to said first ring conformer, and a second plurality of torsions, corresponding to said second ring conformer, said torsions comprising said atoms and bonds in common between said first ring conformer and said second ring conformer;
determining an intersection of said ring conformers, to form node conformers, said intersection comprising conformers having a value of torsion and a value of said indicator of stereo-isomerism in common;
determining an approximate energy level for each node conformer, said energy level comprising a sum of energy levels for individual conformers retrieved from said library;
eliminating from said plurality of node conformers, ones having said approximate energy level exceeding a user-specified energy level to arrive at a preferred node conformer, said preferred node conformer being said conformation of said molecule.

28. The apparatus of claim 27 wherein said torsion comprises a quartet of atoms.

29. The apparatus of claim 27 wherein said intersection further comprises determining a root mean square (RMS) of fit between ring conformers for said atoms and bonds in common.

30. The apparatus of claim 27 wherein said indicator of stereo-isomerism comprises a −1 for counterclockwise and a −2 for clockwise.

31. A method for producing a library of conformers for polycyclic ring analysis of a node, said method comprising:
determining conformers of said node;
determining simple ring conformers from a context of said node, said determining further comprising:
removing stubs;
removing stereochemistry;
removing cis and trans isomerism;
making atoms in the ring an equivalent atom;
determining at least one of a plurality of node conformers based upon said simple ring conformers and said conformers of said node.

32. The method of claim 31 wherein the equivalent atom comprises at least one carbon atom.

33. The method of claim 31 wherein said removing stubs further comprises removing atoms and bonds not comprising the ring.
